(19) United States
(12) Patent Application Publication
(10) Pub. No.: US 2011/0118364 A1
(43) Pub. Date: May 19, 2011

(54) PHARMACEUTICAL COMPOSITIONS OF NANOPARTICLES CONTAINING ACTIVE INGREDIENTS

(76) Inventor: Fabio Moyses LINS DANTAS, Rio de Janeiro (BR)

(21) Appl. No.: 13/002,036

(22) PCT Filed: Jun. 30, 2009
(86) PCT No.: PCT/BR09/00192

§ 371 Date: Dec. 29, 2010

(30) Foreign Application Priority Data
Jun. 30, 2008 (BR) PI0802233-0

(51) Int. Cl. A61K 47/32; C07K 2/00; C07K 14/00; C08B 31/00; C08B 37/08; C08B 37/04; A61K 47/42; A61K 47/36; A61K 47/30; A61K 8/73; A61K 8/85; A61K 8/81; A61K 8/84; A61K 8/64; C08G 63/08; C08G 63/06; C08F 122/32; B29B 9/12; A23D 7/00; A23D 7/04; B82Y 5/00; B82Y 30/00; B82Y 40/00

(52) U.S. Cl. 514/772.4; 530/350; 530/300; 536/102; 536/20; 536/3; 514/773; 514/777; 514/778; 514/779; 514/772.3; 528/272; 528/361; 526/298; 264/5; 426/98; 428/402; 977/773; 977/906; 977/788; 977/795; 977/888

(57) ABSTRACT

This invention concerns a manufacturing process for nanoparticles composted of biodegradable polymers and active ingredients with therapeutic, cosmetic, veterinary, and alimentary applications, and a composition which contains said nanoparticles, which are used in products for animals, including humans. The process consists of emulsifying the hydrosoluble substances to form a w/o emulsion; dissolving the non-emulsionable substances, liposoluble polymer or polymer/compounds in organic solvents; mixing the w/o emulsion and the organic solution of the hydrophobics to form a pre-emulsioned mixture; adding the pre-emulsioned mixture, with the assistance of an injector system, to an aqueous emulsifier solution under ultradispersion to form the final emulsion; leading the final emulsion to evaporation, then centrifuge, freeze, and lyophilize. One variation of the method takes place when the hydrosoluble emulsioned compounds and the liposoluble polymer or polymer/compounds solution are injected separately over the aqueous emulsifying solution. The process of the invention allows nanoparticles of active ingredients to be obtained with a rigorous control over the size of the particle, preserving the active characteristics of the encapsulated compounds.

This invention concerns a manufacturing process for nanoparticles composed of biodegradable polymers and active ingredients for therapeutic, cosmetic, veterinary, and alimentary uses, and also a composition which contains said nanoparticles, which are used in products for animals, including humans.

In recent years, a significant effort has been made to develop the nanotechnology for releasing active ingredients, since this technique offers an adequate means of releasing small particles containing the active ingredient of interest, as well as macromolecules (proteins, peptides, or genes), for vectorized release (PANYAM J.; LABHASETWAR V. Biodegradable Advanced Drug Delivery Reviews, v. 55, p. 329-347, 2003).

The term active ingredient includes any substance with pharmacological, cosmetic, veterinary, and alimentary activity that can be incorporated in the nanoparticles of this invention.

The release agents focused on in nanotechnology are nanoparticles, nanocapsules, nanogels, micellar systems, and conjugates formed by a natural or synthetic polymer that is biocompatible with the organism. These systems provide the targeted release of the drug to tissues or specific cells, in order to improve oral bioavailability, sustain the effect of the ingredients released, make certain active ingredients soluble for intravascular release, as well as increase the stability of the active ingredients against enzymatic degradation (by nucleases and/or proteases), especially of proteins, peptides, and nucleic acids (ALLÉMANN E.; LEROUX J.; GURNY R. Advanced Drug Delivery Reviews, v. 34, p. 171-189, 1998).

The nanometric dimensions of these new systems offer huge advantages for releasing active ingredients. Due to the subcellular and sub-micrometrical sizes, nanoparticles can penetrate deep into tissues through fine capillaries, may cross imperfections existing in the epithelial lining, and are efficiently absorbed by the cells. In addition, by modifying the properties of the polymer used as a matrix, different release modulations of active ingredients can be created, and the structures for specific release sites can be vectorized (PANYAM J.; LABHASETWAR V. Biodegradable Advanced Drug Delivery Reviews, v. 55, p. 329-347, 2003)

The process of endocytosis or phagocytosis of the macrophages is responsible for the efficient release of the active ingredients by means of these new colloidal agents for these cells. The macrophages are widely and strategically distributed in various tissues of the human body with the function of recognizing altered cells and intruder particles, as well as macromolecular ligands in specialized receptor membranes (MOGHIMI S. M.; HUNTER A. C.; MURRAY J. C Pharmacological Reviews, v. 53, n. 2, p. 283-318, 2001.).

Nanoparticles have high cellular absorption rates compared to microparticles (PANYAM J.; LABHASETWAR V. Biodegradable Advanced Drug Delivery Reviews, v. 55, p. 329-347, 2003). Previous studies showed that nanoparticles 100 nm in size showed absorption rates, in Caco-2 cells, that were two and a half times greater compared to microparticles of one (1) μm and six times greater when compared to microparticles of 10 μm. Similar results were obtained when these formulations were tested in a rat intestinal model, showing an absorption rate of 15 to 250 times greater than that presented by the microparticles (PANYAM J.; LABHASETWAR V. Biodegradable Advanced Drug Delivery Reviews, v. 55, p. 329-347, 2003).

Nanoparticles have different names according to the technique used to obtain them; nanocapsules or nanospheres can be obtained. Nanocapsules are nanoparticle carriers composed of an oil core, in which the active ingredient is confined, surrounded by a polymeric membrane containing a hydrophilic or lipofilic surfactant in the interface (NISHIOKA Y; YOSHINO H. Lymphatic target with nanoparticles system. Advanced Drug Delivery Reviews, v. 47, p. 55-64, 2001.). On the other hand, nanospheres are matrices in which the active ingredient is physically dispersed, not necessarily uniformly; however, without the utilization of the oil core (QUINTANAR-GUERRERO D.; ALLÉMANN E.; DOELKER E.; FESSI H. Pharmaceutical Research, v. 15, n. 7, p. 1056-1062, 1998). Nanoparticles are the generic name given to nanospheres and nanocapsules.

As mentioned, a large number of different polymers are used in the production of nanoparticles, which may be of natural or synthetic origin. Included among these polymers are: poly(lactic acid), poly(glycolic), polycaprolactone, alginic acid, chitosan, copolymers, and modified structures of these polymers.

The use of biodegradable synthetic polymers for human transmission began in the 70s, when sutures based on polymers synthesized with lactic acid and glycolic acid were approved by the FDA (Food and Drug Administration) (SUN Y.; WATTS D. C.; JOHNSON J. R. et al. American Pharmaceutical Review, 2001. Available at http://www.americanpharmaceuticalreview.com/past_articles.htm accessed on May 4, 2002). Currently, PLA (poly(lactic acid)), PGA (poly(glycolic acid), and PLGA (poly(lactic acid-co-glycolic)) have a range of applications, used in various areas, such as foods (as films for packages, thickeners, stabilizers), in agriculture, in safety (protective clothing), personal hygiene (sanitary napkins, diapers, creams), among others (VAN VAN DE VELDE K.; KIEKENS P. Polymer Testing, v. 21, p. 433-442, 2002.).

In medicine, the use of these polymers can be divided into three main categories: surgical implants, cicatrizing products, and products for releasing active ingredients. As wound cicatrizants, they are reabsorbed by the skin after the substitution of the injured tissue, same as in sutures, clips, and small parts that are inserted by surgeries (VAN VAN DE VELDE K.; KIEKENS P. Polymer Testing, v. 21, p. 433-442, 2002). Recent studies on the use of sutures using copolymers derived from lactic acid and glycolic acid show that these polymers are not toxic and are completely biodegradable. The synthetic biodegradable polymers are preferable in relation to the natural ones since they are free of immunogenicity and their physical-chemical properties are predictable and reproducible (MOGHIMI S. M.; HUNTER A. C.; MURRAY J. C. Pharmacological Reviews, v. 53, n. 2, p. 283-318, 2001).

This patent request emphasizes the alpha-hydroxy-acids having two and three carbons, since aside from having a wide range of uses in the biomedical area, the derivative polymers have been investigated recently for the release of active ingredients. These polyesters, aside from being biodegradable, are also known as bioabsorbable, since they are hydrolysates when implanted in the organism, forming compatible and metabolizable groupings. The nanoparticles of these polymers are quickly removed from the blood and concentrated in the liver, spleen, and marrow (BRANNON-PEPPAS L. International Journal of Pharmaceutics, v. 116, p. 1-9, 1995.).

The crystallinity of the polymer and the composition of the comonomer also influence biodegradation. The racemic DL polymers, since they are less crystalline than the homopolymers D or L-lactic, are easily broken down, since the amorphous regions are more easily hydrolisated. Polymers of PLGA 50:50 (50% lactic acid and 50% glycolic acid) are broken down more quickly due to the easy hydrolysis of the glycolic acid. The lower the quantity of glycolic acid in the polymer, the slower the biodegradation, since the chain becomes less hydrophilic.

Nanoparticles are prepared by two main methods: conformation of the pre-formed polymers or by the polymerization in situ of the monomer. The polymerization process in situ may be classified in two methods: interfacial and emulsion.

The encapsulation or incorporation from pre-formed polymers is the most widespread technique and can be done using several methods. These techniques are similar to the organic phase, which contains the polymer and the active ingredient, functioning as an internal phase during the process, and the aqueous solution containing a stabilizer, constituting the means of dispersing the nanoparticles. Another similarity between the techniques is the poor efficiency in the encapsulation of the active ingredients of those moderate- to highly-soluble in water, limiting the high yields to lipophilic active ingredients (QUINTANAR-GUERRERO D.; ALLÉMANN E.; DOELKER E.; FESSI H. Drug Development and Industrial Pharmacy, v. 24, n. 12, p. 1113-1128, 1998). The most used techniques are: solvent displacement method, salting-out, emulsification/diffusion method, and emulsification/evaporation of the solvent method.

### Solvent Displacement Method:

This method is a modified version of the solvent evaporation method because it uses water-soluble organic solvents, such as acetone, alcohol, or methanol. Due to the spontaneous diffusion of the solvent in the aqueous phase, an interfacial turbulence is created between the phases, leading to the formation of small particles. The term nanoprecipitation is frequently used to define the process, since the formation of nanoparticles is due to the aggregation of the polymer after the phase changes.

One of the greatest difficulties of this technique is selecting the active ingredient/polymer/solvent/non-solvent system. Each element of this system has a direct influence on the final properties of the nanoparticle.

The polymer concentration, for example, can affect the average diameter as well as the amount of emulsifier in the aqueous phase. The solvent also influences the efficiency of the active ingredient encapsulation; if the active ingredient has no affinity for the solvent, it may migrate to the aqueous phase, resulting in nanoparticles with low amounts of the active ingredient (BODMEIER R.; MCGINITY J. W. International Journal of Pharmaceutical, v. 43, p. 179-186, 1988.).

### Salting-Out:

This method is based on the separation of a solvent miscible in water in the aqueous phase by the salting-out effect. Acetone is the most used miscible solvent since it separates easily from the aqueous phase by the addition of electrolytes. The polymer and the active ingredient are dissolved in an aqueous gel containing a salting-out agent and a colloidal stabilizer. This oil-in-water emulsion is diluted with an adequate volume of water to increase the diffusion of acetone in the aqueous phase, forming the nanoparticles. The solvent and the salting-out agent are eliminated by counterflow filtration. Thus, the diffusion of acetone during the dilution may generate interfacial turbulence and polymer aggregation in the nanoparticles (QUINTANAR-GUERRERO D.; ALLÉMANN E.; DOELKER E.; FESSI H. Drug Development and Industrial Pharmacy, v. 24, n. 12, p. 1113-1128, 1998).

The greatest advantage of this technique is the possibility of incorporating large quantities of the active ingredient in the polymer, generating high yields. Once the solvent/salting-out agent/stabilizer agent system is obtained, it is no longer necessary to look for specific proportions for obtaining nanoparticles (QUINTANAR-GUERRERO D.; ALLÉMANN E.; DOELKER E.; FESSI H. Drug Development and Industrial Pharmacy, v. 24, n. 12, p. 1113-1128, 1998).

On the other hand, the technique is limited to lipophilic active ingredients, salting-out agents that do not precipitate before the separation phase, and soluble stabilizers. Among the salting-out agents that can be used in acetone are saccharose and the electrolytes magnesium chloride, sodium chloride, calcium chloride, and magnesium acetate. As stabilizer agents: PVA, PVP (polyvinylpyrrolidone) and hydroxyethylcellulose have shown good results (QUINTANAR-GUERRERO, 1998).

### Emulsification/Diffusion Method:

This method can be considered a modification of the previous method, without the use of the salting-out agents and a more intense purification. The technique involves the use of a solvent that is partially soluble in water, which is previously saturated in water to guarantee an initial thermodynamic equilibrium in both the liquids. The polymer is dissolved in the solution saturated in water and, in the organic phase, is emulsified under vigorous agitation in an aqueous phase containing a stabilizer. The subsequent addition of water to the system causes the diffusion of the solvent in the external phase, resulting in the formation of the nanoparticles. Depending on the boiling point of the solvent, it is eliminated by distillation or counterflow filtration (QUINTANAR-GUERRERO D.; ALLÉMANN E.; DOELKER E.; FESSI H. Drug Development and Industrial Pharmacy, v. 24, n. 12, p. 1113-1128, 1998).

This technique has some advantages in relation to the others presented, such as the use of less toxic organic solvents, high yields can be obtained, there is high reproducibility from batch to batch, and it is easily scaled up. However, there are some inconveniences, such as the large quantity of water to be eliminated and the diffusion of hydrophilic active ingredients for the external phase during the emulsification, which may result in a reduced encapsulation efficiency.

### Emulsification/Evaporation of the Solvent Method:

This technique is a well-established method based on the classic procedure patented by Vanderhoff et al. In this technique, the polymer is dissolved in an organic solvent, such as dichloromethane or ethyl acetate. The active ingredient is then dissolved or dispersed in the organic solution containing the polymer. This new solution is then introduced in an aqueous solution containing an emulsifier agent (gelatin, albumin, poly(vinyl alcohol)—PVA, polysorbate 80, polyaxamer 188). After the formation of a stable emulsion, the organic phase is evaporated under reduced pressure or continuous agitation. The size control is generally done with the use of an ultrasound.

U.S. Pat. No. 4,272,398 describes a pesticide microencapsulation process, which consists of dissolving the active material by the dissolution of the compound in a biodegradable polymer, polylactic acid, and copolymers of lactic acid and glycolic acid, in a susceptible solvent, methylene chloride, dispersing the solution of the active ingredient and polymer in an aqueous medium, and agitating the dispersion until the solvent evaporates in a way that permits the formation of a capsule for a phase. The aqueous medium consists of water and small quantities of anionic surfactant to help maintain the dispersion. After this phase, some filtering, washing, and drying is still necessary to obtain the microcapsules. However, the document does not have information on the manner of controlling the size of the particle.

Water/oil/water emulsions have also been used to prepare nanoparticles of water-soluble active ingredients (QUINTANAR-GUERRERO D.; ALLÉMANN E.; DOELKER E.; FESSI H. Drug Development and Industrial Pharmacy, v. 24, n. 12, p. 1113-1128, 1998). Although the process seems simple, the technique has many variables which many influence the final product, such as the solubility of the active ingredient and the polymer in the solvent, the type of organic solvent, diffusion rate of the solvent in the aqueous phase, type and concentration of the emulsifier, in addition to later steps for purification (removal of the residual emulsifier) and drying.

Most of the organic solvents used in the process are chlorinated; due to their low solubility in water, they are easily emulsified, solubilize the lipophilic active ingredients well, and also have a low boiling point. However, these solvents are disadvantageous due to their toxicity (most are classified in class 2, by the Guideline of Residual Solvents of the International Conference on Harmonization [ICH]) and should be limited to avoid adverse effects.

Dichloromethane is widely used as a solvent for its low boiling point, which facilitates its later removal from the system, and its low solubility in the aqueous medium, rapidly forming an emulsion. Due to its low solubility in water, dichloromethane forms drops resulting in highly spherical nanoparticles (JULIENNE M. C.; ALONSO M. J.; AMOZA G.; BENOIT J. P. Drug Development and Industrial Pharmacy, v. 18, n. 10, p. 1063-1077, 1992). Water-soluble organic solvents, such as acetone and DMSO (dimethyl sulfoxide), end up forming polymeric agglomerates because of their quick diffusion in the aqueous medium, hindering the formation of nanoparticles (BODMEIER R.; MCGINITY J. W. International Journal of Pharmaceutical, v. 43, p. 179-186, 1988).

Poly(vinyl alcohol) and albumin have been used as stabilizers in aqueous means. PVA provides excellent stabilization in the preparation of the nanoparticles, not only in the emulsification/evaporation method but in all the other techniques. It's one of the few stabilizers that prevents the aggregation of the nanoparticles after preparation (during purification and lyophilization), optimizing the results without the addition of other helpers.

Albumin also is commonly used as a surfactant, replacing PVA. Both the solvent evaporation and the microfluidization appear not to damage the albumin molecules, and the immunogenicity of the albumin absorbed in the nanoparticles is the same as that of a natural solution. However, the source (natural or bovine) and the degree of purity of this macromolecule are aspects which might limit their use.

WO patent no. 2006/109317 shows a preparation process for poly-DL-co-glycolide (PLA) nanoparticles containing drugs for the treatment of tuberculosis. In this patent, the emulsion and the nanoparticles are formed by sonication at low temperatures of 4° C. to 20° C. The nanoparticles formed are centrifuged, washed, and lyophilized.

The type and concentration of the stabilizer are other limiting factors which may affect the size and the polydispersion of the nanoparticles obtained by this technique. Julienne et al. (JULIENNE M. C.; ALONSO M. J.; AMOZA G.; BENOIT J. P. Drug Development and Industrial Pharmacy, v. 18, n. 10, p. 1063-1077, 1992) reported that the nanospheres were obtained with high agitation velocity (10,000 rpm/10 minutes), using 5% p/v of PVA; while, upon using methylcellulose in the same concentration, particles greater than 1 μm were obtained. The authors believe that this difference is due to the greater interfacial reduction of free energy produced by the PVA.

The residual fraction of PVA which remains in the nanoparticles after purification affects the physical properties and the cellular absorption of the final product. Sahoo et al formulated nanoparticles using PLGA 85:15, modifying only the PVA concentration and the type of solvent. It was observed that the organic solvent polarity may affect the quantity of PVA absorbed in the nanoparticles. The more polar the solvent, the greater the quantity of residual PVA. This can be explained by the interaction of the PVA with the polymeric phase, since the organic phase is more miscible with the aqueous (SAHOO S.; PANYAM J.; PRABHA S. et al. Journal of Controlled Release, v. 82, p. 105-114, 2002).

Homogenization of the emulsion is obtained by high-speed mixers (SOPPIMATH K. S.; AMINABHAVI T. M.; KULKARNI A. R.; RUDZINSKI W. E. Biodegradable polymeric nanoparticles as drug delivery devices. Journal of Controlled Release, v. 70, p. 1-20, 2001). The agitation may be mechanical (rotations above 1,000 rpm) or by ultrasound. The homogenization phase is another limiting step in the obtainment of nanoparticles. Cyclosporin A nanospheres were obtained after increasing the homogenization speed. In velocities of 1,000 rpm (for 30 minutes), microparticles approximately 29 μm were obtained; using 10,000 rpm (for 1 minute), nanoparticles of approximately 300 nm were obtained (SÁNCHEZ, 1993).

WO patent 03/099262 describes the emulsification/evaporation of the solvent technique. The document establishes a production process for nanoparticles which includes dissolving a biodegradable polymer in an organic solvent, emulsifying while at the same time doing a sonication and an agitation, and, lastly, isolating and drying the nanoparticle. The active ingredient should be emulsified in such a way that a double emulsion is obtained at the end of the process of the w/o/w type. The proposed method is basically limited to proteins and peptides. The process proposed in this patent provides for a modification of the emulsification process where a high homogeneity of the nanoparticles is obtained by the simultaneous use of a mechanical agitation at high shear (between 4,000 and 15,000) and sonication (frequency of 20 to 70 kHz). However, this system does not permit a rigorous control over the size of the particle, which is defined by various variables, such as the concentration of the emulsifier, the water/organic solvent system, temperature, and nature of the substances contained in the nanoparticles. The size control of the particles is fundamental for defining the penetration power to the tissues and their depuration by the renal and immunological system. For example, particles less than 40 nm may reach the lymphatic system and accumulate in this area.

U.S. Pat. No. 602,004 reveals a process for obtaining protein microparticles, which consists of dissolving the polymer (PLGA) in an organic solvent to obtain a polymeric solution; adding the active ingredient (which may be in the form of an aqueous solution, suspension, or powder) to form the first emulsion or suspension within a continuous phase to produce a dispersion; adding an excipient to produce the final dispersion; freezing and lyophilizing directly to remove the different solvents (aqueous and organic) and obtain the microparticles of proteins for controlled release.

The system proposed here concerns a modification in the emulsification/evaporation technique, and overcomes limiting factors and other deficiencies inherent to the state of the technique by the invention of a manufacturing process for nanoparticles in which it is possible to control the size of the particles. In a paper written by Song et al (Colloids and Surfaces A: physicochem. Eng. Aspects. 276, 2006, 162-167), the author indicates the physical-chemical grounds for operationalizing the size of the particles. Thus, we see now that ionic emulsifiers permit smaller particles because they better stabilize the particles of the dispersed organic solvents. In turn, the organic solvent used needs to have low hydrophobicity in order to minimize the aggregation of the droplets. Controlling these parameters can vary the size of these particles plus the initial energy of a high mechanical shear system (ultradispersion) working above 14,000 rpm. The high-shear mechanical energy is important, though not fundamental for the stabilization of the particle size. However, submicrometric particle sizes are obtained from rotations between 11,000 to 22,000 rpm. Lower and/or higher rotations tend to form particles with large and/or thick granulometric distribution.

Thus, the systems here described are modulated based on these concepts, one of the main differentiators in terms of the proposals of the state of the technique.

### INVENTION SUMMARY

The goal of this invention is to provide a preparation process for nanoparticles containing one or more hydrosoluble and liposoluble active ingredients, preserving the active characteristics of the encapsulated compounds.

Another goal for this invention is to promote a manufacturing method for nanoparticles capable of maintaining rigorous control over the size of the particle.

Yet another goal for the invention is to promote a pharmaceutical, cosmetic, or food composition containing the nanoparticles obtained by the invention process and biologically acceptable vehicles.

The goal of the invention is to provide the use of the nanoparticles obtained following the invention process for pharmaceutical, cosmetic, or food applications.

This and other similar goals, advantages, and characteristics of the invention will become clearer throughout the detailed description of the invention.

### BRIEF DESCRIPTION OF THE FIGURES

FIG. 1—Electron micrograph scan of nanoparticles obtained by the method proposed using PVA as an emulsifier.

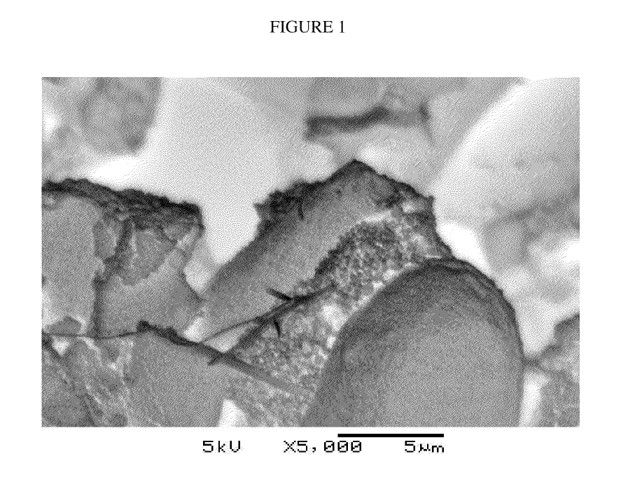

### DETAILED DESCRIPTION OF THE INVENTION

This invention concerns a manufacturing process for nanoparticles containing improved pharmaceutical properties. Generally speaking, the method revealed here provides a manufacturing process for bioabsorbable polymer nanoparticles able to incorporate hydrophilic and lipofilic substances and obtain high-stability nanoparticles.

The nanoparticles obtained by this process may contain one or more substances in one same particle, according to their application. However, it can also contain hydrophilic and lipophilic substances in differentiated particles.

The manufacturing process for nanoparticles employs the method for emulsification/vaporization of organic solvents and uses bioabsorbable polymers to incorporate the compounds. Small variations in the process may occur according to the characteristics of the substance to be incorporated. The hydrosoluble compounds should be previously emulsified to form a water-in-oil (w/o) emulsion. The emulsion uses emulsifiers common to the technique—preferably poly(vinyl alcohol), polyvinylpyrrolidone, lecithin, gelatin, albumin, didodecyl dimethyl ammonium bromide, among others; most preferably, poly(vinyl alcohol), lecithin, and albumin.

The non-emulsionable substances, liposoluble polymer or polymer/compounds, are dissolved in low-toxicity class 2 and 3 organic solvents. Appropriate organic solvents include, but are not limited to, dichloromethane, acetone, ethanol, ethyl acetate, among others; preferably, ethyl acetate and dichloromethane. The amount of solvent used depends on the chemical nature of the substances which form the nanoparticle, and may vary between 1 to 50% v/v. This solution of non-emulsioned substances is, then, placed in an ultrasound and, next, agitated during a period sufficient for its solubilization.

It is important to stress that for an effective imprisonment of the active ingredients, the polymers and the simple substance (emulsioned hydrophilic or hydrophobic) should generate a final system where the (1:1) to (1:10) proportion is found, preferably in the (1:1) proportion. The exact composition of the imprisoned polymer/substance is dependent on the chemical nature of the substance and the desirable characteristics of kinetic release.

Polymers able to be used in this invention include bioabsorbable and natural polymers. For example, poly(lactic acid) and copolymers, poly(glycolic acid) and copolymers, poly-β-hydroxybutyrate acid, polyhydroxyvalerate acid, polyesteramides, polycyanoacrylate, poly(amino acids), polyanhydrides, polyanhydrides, alginate, chitosan, starch, among others. In particular, poly(lactic acid) and copolymers are desirable.

The average molecular weight or viscometry of these polymers may vary between 2,000 to 1,000,000. Preferably, in the case of the poly(lactic acid) and copolymers, from 10,000 to 200,000, and for the PVA and PVP, from 1,000 to 20,000. The copolymers of lactic acid and glycolic acid and isomers are important for the formation of the nanoparticles and lend them versatility in terms of the speed of biodegradation and, consequently, the release of drugs. The preferable molar compositions for lactic acid and glycolic acid are 5 to 95%.

Concomitantly, a emulsifying solution is prepared. Emulsifiers which may be used in the invention include poly(vinyl alcohol), polyvinylpyrrolidone, carboxymethylcellulose, lecithin, gelatin, albumin, non-ionic surfactants such as polyoxyethylene sorbitan fatty acid esters (Tween 80, Tween 60, etc.), anionic surfactants (didodecyl dimethyl ammonium bromide, sodium lauryl sulfate, sodium stearate, etc.), among others. These emulsifiers may be used both together and separately. The emulsifier concentration may vary between 0.01 to 20% p/v; preferably, between 0.1 to 5% p/v.

Lastly, the emulsification processing takes place using an ultradisperser. The emulsified liposoluble polymer/compounds or hydrosoluble polymer and compounds solution are previously mixed and then are injected by means of needles, calibers between .5 to 2 mm, over an aqueous solution with an emulsifier. The dispersion should take place at a speed between 11,000 to 22,000 rpm.

One variation of the method occurs when the emulsified hydrosoluble compounds and the liposoluble polymer/compound solution or polymer are injected separately over the emulsifying aqueous solution.

Anti-foaming agents should be used in order to facilitate dispersion and enable the imprisonment of the nanoparticles, such as alcohols in general, mineral salts, and silicone oil derivatives.

After the ultradispersion, the system is led to evaporation to remove the organic solvent and centrifuged. Evaporation may take place in a rotating evaporator at an evaporation rate of the organic solvent from 0.1 to 40 g/hours.

The decanted material is frozen and lyophilized, obtaining the nanoparticle in a way that may be incorporated to the pharmaceutical formulations for oral, parenteral (subcutaneous, intramuscular, and intravenous), sublingual, rectal, transdermic, inhalation, ophthalmic, and otologic administration. The nanoparticles may also be used in cosmetic, veterinary, and food formulations.

The therapeutic agents may be selected from a variety of known active ingredients, such as, but not limited to: analgesics, anesthetics, analeptics, adrenergic agents, adrenergic blocking agents, adrenolytics, adrenocorticoids, adrenomimetics, anticholinergic agents, anticholinesterasic, anticonvulsants, alkylating agents, alkaloids, allosteric inhibitors, anorexics, antacids, antidiarrheals, anabolic steroids, antidotes, antifolics, antipyretics, antirheumatic agents, psychotherapeutic agents, neural blocking agents, anti-inflammatories, antihelminthics, antiarrhythmic agents, antibiotics, anticoagulants, antidepressants, agents for diabetes, antiepileptics, antifungals, antihistamines, antihypertensive agents, antimuscarinic agents, antimycobacterials, antibacterials, antimalarials, antiseptics, antineoplastic agents, antiprotozoal agents, immunosuppressants, immunostimulants, antireoidal agents, antiviral agents, anxiolytics, sedatives, astringents, β-blocking agents, contrast media, corticosteroids, cough suppressants, diagnostic agents, image diagnostic agents, diuretics, dopaminergics, hemostatics, hematological agents, hemoglobin modifiers, hormones, hypnotics, antihyperlipidemics and other lipid regulators, muscarinics, muscle relaxants, parasympathomimetics, prostagladins, radiopharmaceuticals, sedatives, antiallergics, stimulants, sympathomimetic, thyroid agents, vasodilators, vaccines, vitamins and xanthines, antineoplastics and anti-cancer agents. The therapeutic agents may be biological, such as: proteins (ex.: enzymes and antibodies), polypeptides, carbohydrates, polynucleotides, and nucleic acids. The medicines (pharmaceutical compositions) may be produced by known techniques.

Cosmetic agents are considered as: any active ingredient that has cosmetic action; they are also able to be incorporated to the nanoparticles of this invention. Examples of these ingredients include emmolients, humectants, free-radical inhibitor agents, anti-inflammatories, vitamins, depigmenting agents, anti-acne, antiseborrheics, keratolytics, skin-coloring agents, fat-reducing agents, and antioxidants. The cosmetics may be prepared using known techniques.

Examples of food applications include, but are not limited to: encapsulation of proteins, carbohydrates, hydrosoluble and liposoluble vitamins, and other food supplements. The food supplements may be produced using known techniques.

The size of the nanoparticles obtained by the process of the invention vary from 20 to 500 nm and are measured by microscopic image analysis, zeta potential, or light diffraction.

Complemented, the proposed system is superior to the state of the technique because it uses the ultradispersion system and not the sonication system. The latter does not allow for a rigorous control over particle sizes. In addition, these and other patents do not take into account important parameters, such as: evaporation speed of the organic solvent, pre-emulsion of the hydrosoluble active ingredients, and control over the particle size by water concentration/organic solvent/emulsifier/and injection needle diameter ratios. Only by controlling these parameters can the encapsulation capacity (or imprisonment), quality, size, size distribution, and morphology of the particles be controlled.

Following are some merely illustrative examples of the invention which in no way limit the scope of protection of this invention.

### EXAMPLES

### Method 1

Pre-emulsion and mixture: The hydrosoluble substances should be weighed and dissolved in 10 ml of a 0.1% PVP solution and left under agitation for 12 hours. Next, this solution should be injected into 90 ml of dichloromethane over the vortex of the ultradisperser at 14,000 rpm and the system should be left under agitation for 5 minutes. Note the dispersion temperature with a calibrated thermometer. Measure the conductance and the pH of the dispersion (wait for it to stabilize). Concomitantly, the hydrophobic and solubilized substances should be weighed in 10 ml of dichloromethane. The w/o emulsion of the hydrophilic and the organic solution of the hydrophobics should be mixed at the end.

Emulsification processing: An ultradisperser was used to prepare the final emersion, spinning at 14,000 rpm. In a 300 ml beaker, 150 ml of the 5% PVP solution was added. The pre-emulsioned mixture was added to the agitation vortex with a syringe whose needle had an internal diameter of approximately 1 mm. Simultaneously, the emulsion should be added containing the hydrosoluble active ingredients. Absolute ethanol was used with an anti-foaming agent to facilitate the dispersion and enable the imprisonment of active ingredients in the nanoparticles. Next, the system went to a rotating evaporator to remove the organic solvent at 10 r/hour and centrifuged. The decanted material is frozen for 24 hours and lyophilized next. Particles with dimensions between 200-500 nm are obtained.

### Method 2

Pre-emulsion and mixture: The hydrosoluble substances should be weighed and dissolved in 10 ml of the 0.2% solution of didodecyl dimethyl ammonium (BDDA) and left under agitation for 12 hours. Next, this solution should be injected into 90 ml of ethyl acetate saturated in water over the vortex of the ultradisperser at 22,000 rpm, and the system should be left under agitation for 5 minutes. Note the temperature of the dispersion with a calibrated thermometer. Measure the conductance and the pH of the dispersion (wait for it to stabilize). Concomitantly, the hydrophobic and solubilized substances should be weighed in 10 ml of ethyl acetate saturated with water. The w/o emulsion of the hydrophilics and the organic solution of the hydrophobics should be mixed mechanically at the end.

Emulsification processing: An ultradisperser spinning at 22,000 rpm was used to prepare the emulsion. In a 300 ml beaker, 150 ml of the 0.2% BDDA solution was added. A pre-emulsioned mixture was added to the agitation vortex with a syringe whose needle had an internal diameter of approximately 1 mm. Absolute ethanol was used with an anti-foaming agent to facilitate the dispersion and enable the imprisonment of the active ingredients in the nanoparticles. Next, the system went to a rotating evaporator for the removal of the organic solvent at 40 r/hour and centrifuged. The decanted material is frozen and lyophilized. Particles with dimensions between 40 to 150 mm are obtained.

### Method 3

Pre-emulsion and mixture: The hydrosoluble substances should be weighed in 10 ml of a 0.5% lecithin solution and left under agitation for 12 hours. Next, this solution should be injected to 90 ml of dichloromethane over the vortex of the ultradisperser at 14,000 rpm, and the system should be left under agitation for 5 minutes. Note the temperature of the dispersion with a calibrated thermometer. Measure the conductance and the pH of the dispersion (wait for it to stabilize). Concomitantly, the hydrophobic and solubilized substances should be weighed in 10 ml of dichloromethane. The w/o emulsion of the hydrophilics and the organic solution of the hydrophobics should be mixed mechanically at the end.

Emulsification processing: An ultradisperser spinning at 14,000 rpm was used to prepare the emulsion. In a 300 ml beaker, 150 ml of the 0.5% lecithin solution was added to the agitation vortex with a syringe whose needle had an internal diameter of approximately 1 mm. Next, the system went to a rotating evaporator to removal the organic solvent at 10 r/hour and centrifuged. The decanted material is frozen and lyophilized. Particles with dimensions between 50-200 nm are obtained.

### Method 4

Pre-emulsion and mixture: The hydrosoluble substances should be weighed and dissolved in 10 ml of a 0.2% didodecyl dimethyl ammonium (BDDA) solution and left under agitation for 12 hours. Next, this solution should be injected into 90 ml of ethyl acetate saturated with water over the vortex of the ultradisperser at 22,000 rpm, and the system should be left under agitation for 5 minutes. Note the temperature of the dispersion with a calibrated thermometer. Measure the conductance and the pH of the dispersion (wait for it to stabilize). Concomitantly, the hydrophobic substances should be weighed and solubilized in 10 ml of dichloromethane. The w/o emulsion of the hydrophilics and the organic solution of the hydrophobics should be mixed mechanically at the end.

Emulsification processing: An ultradisperser spinning at 22,000 rpm was used to prepare the emulsion. In a 300 ml beaker, 150 ml of the 0.2% lecithin solution was added. The pre-emulsioned mixture was added to the agitation vortex with a syringe whose needle had an internal diameter of approximately 1 mm. Absolute ethanol was used with an anti-foaming agent to facilitate the dispersion and enable the imprisonment of the active ingredients in the nanoparticles. Next, the system went to a rotating evaporator to remove the organic solvent at 40 r/hour and centrifuged. The decanted material is frozen and lyophilized. Particles with dimensions between 50-300 nm are obtained.

### Method 5

Pre-emulsion and mixture: The hydrosoluble substances should be weighed and dissolved in 10 ml of a 5% PVA solution and left under agitation for 12 hours. Next, this solution should be injected into 90 ml of dichloromethane over the vortex of the ultradisperser at 14,000 rpm, and the system should be left under agitation for 5 minutes. Note the temperature of the dispersion with a calibrated thermometer. Measure the conductance and the pH of the dispersion (wait for it to stabilize). Concomitantly, the hydrophobic substances should be weighed and solubilized in 10 ml of dichloromethane. The w/o emulsion of the hydrophilics and the organic solution of the hydrophobics should be mixed mechanically at the end.

Emulsification processing: An ultradisperser spinning at 14,000 rpm was used to prepare the emulsion. In a 300 ml beaker, 150 ml of the 5% PVA solution was added. The pre-emulsioned mixture was added to the agitation vortex with a syringe whose needle had an internal diameter of approximately 1 mm. Absolute ethanol was used with an anti-foaming agent to facilitate the dispersion and enable the imprisonment of the active ingredients in the nanoparticles. Next, the system was sent to an rotating evaporator to remove the organic solvent at 10 r/hour and centrifuged. The decanted material is frozen and lyophilized. Particles with dimensions between 50-300 nm are obtained.

1. PREPARATION PROCESS OF THE NANOPARTICLES, characterized by the fact that it includes the following stages:
a) emulsify the hydrosoluble compounds to form a w/o emulsion;
b) dissolve the non-emulsionable substances, liposoluble polymer or polymer/compounds in low-toxicity class 2 and 3 organic solvents;
c) mix the w/o emulsion and the organic solution of the hydrophobics to form the pre-emulsioned mixture;
d) add the pre-emulsioned mixture with the assistance of an injector system to an aqueous emulsifier solution under ultradispersion to form the final emulsion;
e) Lead the final emulsion to evaporation, then centrifuge, freeze, and lyophilize.

2. PREPARATION PROCESS OF THE NANOPARTICLES, in accordance with claim 1, characterized by the fact that the emulsifiers used to form the w/o emulsion are selected from poly(vinyl alcohol), polyvinylpyrrolidone, lecithin, gelatin, albumin, and didodecyl dimethyl ammonium bromide.

3. PREPARATION PROCESS OF THE NANOPARTICLES, in accordance with claim 2, characterized by the fact of the emulsifier being poly(vinyl alcohol), lecithin, or albumin.

4. PREPARATION PROCESS OF THE NANOPARTICLES, in accordance with claims 1 to 3, characterized by the fact of the class 2 and 3 organic solvents being dichloromethane, acetone, ethanol, or ethyl acetate.

5. PREPARATION PROCESS OF THE NANOPARTICLES, in accordance with claim 1, characterized by the fact that the quantity of class 2 and 3 organic solvents are within the 1 to 50% p/v range.

6. PREPARATION PROCESS OF THE NANOPARTICLES, in accordance with claims 1 to 5, characterized by the fact that the polymers are selected from poly(lactic acid) and copolymers, poly(glycolic acid) and copolymers, poly-β-hydroxybutyrate acid, polyhydroxyvalerate acid, polyesteramides, polycyanoacrylate, poly(amino acids), polyanhydrides, polyanhydrides, alginate, chitosan, and starch.

7. PREPARATION PROCESS OF THE NANOPARTICLES, in accordance with claims 1 to 6, characterized by the fact that the average molecular weight or viscometric of these polymers may vary between 2,000 to 1,000,000.

8. PREPARATION PROCESS OF THE NANOPARTICLES in accordance with claim 6, characterized by the fact that the polymer is the poly(lactic acid) and its copolymers.

9. PREPARATION PROCESS OF THE NANOPARTICLES, in accordance with claim 8, characterized by the fact that the average molecular weight or viscometric of the poly(lactic acid) and its copolymers are within the 10,000 to 200,000 range.

10. PREPARATION PROCESS OF THE NANOPARTICLES, in accordance with claims 1 to 7, characterized by the fact that the copolymers of lactic acid and glycolic acid are present in the molar compositions, which vary between 5 to 95%.

11. PREPARATION PROCESS OF THE NANOPARTICLES, in accordance with claims 1 to 10, characterized by the fact that the relation between the polymer and the imprisoned substance, emulsioned hydrophilic or hydrophobic, is of 1:1 to 1:10.

12. PREPARATION PROCESS OF THE NANOPARTICLES, in accordance with claim 11, characterized by the fact that the relation between the polymer and the imprisoned substance, emulsioned hydrophilic or hydrophobic, is of 1:1.

13. PREPARATION PROCESS OF THE NANOPARTICLES, in accordance with claims 1 to 12, characterized by the fact that the aqueous emulsifier solution uses the emulsifiers poly(vinyl alcohol), polyvinylpyrrolidone, carboxymethylcellulose, lecithin, gelatin, albumin, non-ionic surfactants such as polyoxyethylene sorbitan fatty acid esters (Tween 80, Tween 60), and anionic surfactants (didodecyl dimethyl ammonium bromide, sodium lauryl sulfate, sodium stearate) together or separately.

14. PREPARATION PROCESS OF THE NANOPARTICLES, in accordance with claim 13, characterized by the fact that the concentration of the emulsifier is within the 0.01 to 20% p/v range.

15. PREPARATION PROCESS OF THE NANOPARTICLES, in accordance with claim 14, characterized by the fact that the concentration of emulsifiers is within the 0.1 to 5 p/v range.

16. PREPARATION PROCESS OF THE NANOPARTICLES, in accordance with claims 1 to 15, characterized by the fact that the injector system has needles of .5 to 2 mm calibers.

17. PREPARATION PROCESS OF THE NANOPARTICLES, in accordance with claims 1 to 16, characterized by the fact that the dispersion of the final emulsion is done at a speed of 11,000 to 22,000 rpm.

18. PREPARATION PROCESS OF THE NANOPARTICLES, in accordance with claims 1 to 17, characterized by the fact that anti-foaming agents are also used in the aqueous emulsifying solution.

19. PREPARATION PROCESS OF THE NANOPARTICLES, in accordance with claim 18, characterized by the fact that the anti-foaming agents are selected from alcohols, mineral salts, and silicone oil derivatives.

20. PREPARATION PROCESS OF THE NANOPARTICLES, in accordance with claim 19, characterized by the fact that the anti-foaming agent is ethanol.

21. PREPARATION PROCESS OF THE NANOPARTICLES, in accordance with claims 1 to 20, characterized by the fact that the evaporation may be done in a rotating evaporator at a evaporation speed of the organic solvent of 0.1 to 40 r/hour.

22. PREPARATION PROCESS OF THE NANOPARTICLES, characterized by the fact that it includes the following stages:
a) emulsify the hydrosoluble compounds to form a w/o emulsion;
b) dissolve the non-emulsionable substances, liposoluble polymer or polymer compounds in low-toxicity class 2 and 3 organic solvents;
c) simultaneously add the w/o emulsion and the organic solution of the hydrophobics (b) with the assistance of the injector systems to an emulsifying aqueous solution under ultradispersion to form the final emulsion;
e) Lead the final emulsion to evaporation, then freeze and lyophilize.

23. PREPARATION PROCESS OF THE NANOPARTICLES, in accordance with claim 22, characterized by the fact that the emulsifiers used to form the w/o emulsion are selected from poly(vinyl alcohol), polyvinylpyrrolidone, lecithin, gelatin, albumin, and didodecyl dimethyl ammonium bromide.

24. PREPARATION PROCESS OF THE NANOPARTICLES, in accordance with claim 23, characterized by the fact that the emulsifier is poly(vinyl alcohol), lecithin, or albumin.

25. PREPARATION PROCESS OF THE NANOPARTICLES in accordance with claims 22 to 24, characterized by the fact that the class 2 or 3 organic solvents are dichloromethane, acetone, ethanol, or ethyl acetate.

26. PREPARATION PROCESS OF THE NANOPARTICLES, in accordance with claim 25, characterized by the fact that the amount of class 2 and 3 organic solvents is within the 1 to 50% p/v range.

27. PREPARATION PROCESS OF THE NANOPARTICLES, in accordance with claims 22 to 26, characterized by the fact that the polymers are selected from poly(lactic acid) and copolymers, poly(glycolic acid) and copolymers, poly-β-hydroxybutyrate acid, polyhydroxyvalerate acid, polyesteramides, polycyanoacrylate, poly(amino acids), polyanhydrides, polyanhydrides, alginate, chitosan, and starch.

28. PREPARATION PROCESS OF THE NANOPARTICLES, in accordance with claim 27, characterized by the fact that the polymer is poly(lactic acid) and its copolymers.

29. PREPARATION PROCESS OF THE NANOPARTICLES, in accordance with claims 22 to 28, characterized by the fact that the relation between polymer and imprisoned substance, emulsioned hydrophilic or hydrophobic, is of 1:1 to 1:10.

30. PREPARATION PROCESS OF THE NANOPARTICLES, in accordance with claim 29, characterized by the fact that the relation between polymer and imprisoned substance, emulsioned hydrophilic or hydrophobic, is of 1:1.

31. PREPARATION PROCESS OF THE NANOPARTICLES, in accordance with claims 22 to 30, characterized by the fact that the emulsifier aqueous solution uses the emulsifiers poly(vinyl alcohol), polyvinylpyrrolidone, carboxymethylcellulose, lecithin, gelatin, albumin, non-ionic surfactants such as polyoxyethylene sorbitan fatty acid esters (Tween 80, Tween 60), and anionic surfactants (didodecyl dimethyl ammonium bromide, sodium lauryl sulfate, sodium stearate), together or separately.

32. PREPARATION PROCESS OF THE NANOPARTICLES, in accordance with claim 31, characterized by the fact of the concentration of the emulsifier being within the 0.01 to 20% p/v range.

33. PREPARATION PROCESS OF THE NANOPARTICLES in accordance with claim 32, characterized by the fact that the concentration of the emulsifier is within the 0.1 to 5 p/v range.

34. PREPARATION PROCESS OF THE NANOPARTICLES, in accordance with claims 22 to 33, characterized by the fact that the injector system has needles of .5 to 2 mm calibers.

35. PREPARATION PROCESS OF THE NANOPARTICLES, in accordance with claims 22 to 34, characterized by the fact that the dispersion of the final emulsion is done at a speed of 11,000 to 22,000 rpm.

36. PREPARATION PROCESS OF THE NANOPARTICLES, in accordance with claims 22 to 35, characterized by the fact that anti-foaming agents are also used in the emulsifying aqueous solution.

37. PREPARATION PROCESS OF THE NANOPARTICLES, in accordance with claim 36, characterized by the fact that the anti-foaming agents are selected from alcohols, mineral salts, and silicone oil derivatives.

38. PREPARATION PROCESS OF THE NANOPARTICLES, in accordance with claim 37, characterized by the fact that the anti-foaming agent is ethanol.

39. PREPARATION PROCESS OF THE NANOPARTICLES, in accordance with claims 22 to 38, characterized by the fact that the evaporation may take place in a rotating evaporator at a speed of evaporation of the organic solvent at 0.1 to 40 r/hour.

40. NANOPARTICLES, characterized by the fact that they are obtained according to the processes in claims 1 to 21.

41. NANOPARTICLES, characterized by the fact that they are obtained according to the processes in claims 22 to 39.

42. NANOPARTICLES, in accordance with claims 40 and 41, characterized by the fact of being within 20 to 500 nm in size.

43. USE OF NANOPARTICLES, characterized by the fact of being obtained according to the processes in claims 1 to 21 and/or 22 to 39 for pharmaceutical, cosmetic, and food applications.

44. PHARMACEUTICAL FORMULATION, characterized by the act that it contains the nanoparticles obtained in accordance with the processes in claims 1 to 21 and/or 22 to 39 and pharmaceutically-acceptable excipients.

45. COSMETIC FORMULATION, characterized by the fact that it contains the nanoparticles obtained according to the process in claims 1 to 21 and/or 22 to 39 and acceptable excipients.

46. FOOD FORMULATION, characterized by the fact that it contains the nanoparticles obtained in accordance with the processes in claims 1 to 21 and/or 22 to 39 and biologically-acceptable excipients.

47. PRODUCT, characterized by the fact that it contains nanoparticles obtained according to the processes in claims 1 to 21 and/or 22 to 39.
